(19) United States
(12) Patent Application Publication
(10) Pub. No.: US 2009/0247879 A1
(43) Pub. Date: Oct. 1, 2009

(54) Extended ultrasound imaging probe for insertion into the body

(76) Inventors: Bjorn A.J. Angelsen, Trandheim (NO); Tonni F. Johansen, Trondheim (NO)

Correspondence Address: COHEN, PONTANI, LIEBERMAN & PAVANE LLP, 551 FIFTH AVENUE, SUITE 1210, NEW YORK, NY, 10176, US

(21) Appl. No.: 12/381,728

(22) Filed: Mar. 16, 2009

Related U.S. Application Data
(63) Continuation-in-part of application No. 11/077,521, filed on Mar. 10, 2005, now abandoned.
Application No. 11/077,521 is a continuation-in-part of application No. 11/075,929, filed on Mar. 9, 2005.
(60) Provisional application No. 60/551,736, filed on Mar. 10, 2004.
(60) Provisional application No. 60/551,681, filed on Mar. 9, 2004.

(51) Int. Cl. A61B 8/14

(52) U.S. Cl. 600/463

(57) ABSTRACT

An ultrasound imaging probe for real time 3D ultrasound imaging from the tip of the probe that can be inserted into the body. The ultrasound beam is electronically scanned within a 2D azimuth plane with a linear array, and scanning in the elevation direction at right angle to the azimuth plane is obtained by mechanical movement of the array. The mechanical movement is either achieved by rotation of the array through a flexible wire, or through wobbling of the array, for example through hydraulic actuation. The probe can be made both flexible and stiff, where the flexible embodiment is particularly interesting for catheter imaging in the heart and vessels, and the stiff embodiment has applications in minimal invasive surgery and other procedures. The probe design allows for low cost manufacturing which allows factory sterilized probes to be disposed after use.

### CROSS REFERENCE TO RELATED APPLICATIONS

This application is a continuation-in-part of U.S. patent application Ser. No. 11/077,521 which was filed with the U.S. Patent and Trademark Office on Mar. 10, 2005, which is based on U.S. Provisional Patent Application Ser. No. 60/551,736, filed Mar. 10, 2004

This application is also a continuation-in-part of U.S. patent application Ser. No. 11/075,929 which was filed on Mar. 9, 2005, which is based on U.S. Provisional Patent Application Ser. No. 60/551,681, filed Mar. 9, 2004.

The entire contents of each of the above applications is expressly incorporated herein by reference.

### BACKGROUND OF THE INVENTION

1. Field of the Invention

The invention relates to methods, ultrasound probes, and instrumentation for real time 3D imaging around the tip of an ultrasound probe that can be inserted into the body, either through natural openings or through surgical wounds.

2. Description of the Related Art

Real time (Rt) two-dimensional (2D) and three-dimensional (3D) ultrasound imaging around the tip of a flexible ultrasound probe is in many situations a sought after tool, both for diagnosis and for guidance of procedures. Examples of such procedures are placement of devices in the heart ventricles and atria, guidance of electrophysiology ablation, or guidance in minimal invasive surgery. In these cases, the ultrasound probe gets in direct contact with the blood path, and it is then a great advantage to use factory-sterilized, disposable probes. This requires that the manufacturing cost of the probes can be kept low.

Such imaging around a probe tip is done by lateral scanning of a pulsed ultrasound beam from the probe tip, where 3D imaging requires scanning in two lateral directions within a volume, while 2D imaging is obtained by lateral scanning in one direction across a surface. The volume scanning is ideally done with a two-dimensional matrix array where the element width is less than λ/2 in both directions, where λ is the wave-length of the ultrasound in the tissue. However this gives a large amount of small elements, that either requires an impractically thick cable connecting the array and the instrument, or requires a large amount of beam forming electronics at the probe tip close to the array, which is expensive and space consuming to be used with probes that are inserted into the body. The small size of the elements of the matrix probe also makes it difficult to manufacture the matrix arrays for ultrasound frequencies above ˜5 MHz.

There is further a need for the probe to be flexible, for example for insertion into the vessels and the heart as a catheter. In this situation one could also want to control flexing of the tip from the external instrument. In other situations, like endoscopic surgery, one would like to have a stiff probe.

### SUMMARY OF THE INVENTION

The present invention provides a solution to these problems by using mechanical direction steering of the ultrasound beam in one direction, and for 3D imaging the ultrasound beam is electronically scanned in the 2^{nd} direction. For 2D imaging one can mechanically direction scan a single element transducer with a fixed focus, or an annular array transducer to obtain electronically steered dynamic focusing. For the annular array one can conveniently use solutions as described in U.S. Pat. No. 6,540,677 to increase the sensitivity and reduce the number of wires connecting between the probe tip and the external imaging instrument. For 3D imaging the electronic bean scanning in the 2^{nd} direction can be obtained by a linear phased, curvilinear switched, or linear switched array. Example embodiments of the mechanical direction scanning of the beam are shown by rotation of the array by a flexible cable through the probe, or mechanical wobbling of the array, for example by hydraulic means.

For limited movement velocity of the imaging object, one can obtain depth variable focusing (i.e. dynamic focusing), both of the transmit and the receive beam, of the ultrasound beam in the mechanical scan direction by linear combination of the received RF signal from neighboring mechanical scan directions. For 2D imaging, one can also obtain electronic steering of the focus symmetrically around the beam axis with an annular array as described above. For 3D imaging one obtains electronic steering of the focus in the electronic scan direction (referred to as the azimuth direction) with the electronic array beam former. In the mechanical scan direction (referred to as the elevation direction) one can then for slow moving objects obtain depth variable focusing by linear combination of the received RF-signal from neighboring beams in the elevation direction. 3D electronic steering of the beam focus in the elevation direction can also be obtained by dividing the array elements in the elevation direction, with for example a switched aperture focusing, or a steerable or switchable signal delay based focusing. To minimize the number of wires that connect the array and the external imaging instrument, the elevation focusing can be done by electronics at the tip of the probe, to reduce the number of wires connecting the tip of the imaging probe and the external imaging instrument. Sub-aperture beam forming in the azimuth direction by electronics at the tip of the probe, also provides a reduction in the number of wires connecting the imaging probe tip and the external imaging instrument.

The probes can be made both flexible and stiff, for best adaption to the application. The tip of the flexible probe can be direction steered (flexed) through wires along the periphery of the probe that are stretched/released through handles at the outside instrument.

### BRIEF DESCRIPTION OF THE DRAWINGS

In the drawings:

FIG. 1 shows an overview of a real time 2D or 3D imaging system with a probe according to the invention, and

FIG. 2 shows an example embodiment of the distal tip of a flexible, probe according to the invention where the mechanical beam scanning is obtained by a mechanical rotation of the array, and

FIG. 3 shows examples of three types of arrays for electronic azimuth scanning of the beam with their corresponding image formats for 3D imaging, and

FIG. 4 shows an example of a 3D region that can be scanned by an ultrasound beam from the distal probe tip of the type shown in FIG. 2, and

FIG. 5 shows yet another arrangement of 3D imaging with a rotating array according to the invention, and

FIG. 6 shows an example 2D display of the conic image on a screen, and

FIG. 7 shows yet another arrangement with two rotating transducers according to the invention, and

FIG. 8 shows a method of combining the element signals from a sub-aperture group of neighboring elements, to reduce the number of signals that must be connected to the imaging instrument, and

FIG. 9 shows example transmitted beam profiles with grating lobes that is found with transmitting with less than all array elements in a sub-aperture arrangement, and

FIG. 10 shows yet another method of mechanical scanning of the ultrasound beam from the distal tip of the probe, according to the invention, and

FIG. 11 shows an example of a 3D region that can be scanned with an ultrasound beam generated by the arrangement in FIG. 10, and

FIG. 12 shows an example of an optical angular position resolver for measuring the mechanical rotation of the array in a probe tip like displayed in FIG. 2, and

FIG. 13 shows an example of an optical angular position resolver for measuring the angular wobbling of the array for a probe tip of the type shown in FIG. 10.

### DETAILED DESCRIPTION OF THE PRESENTLY PREFERRED EMBODIMENTS

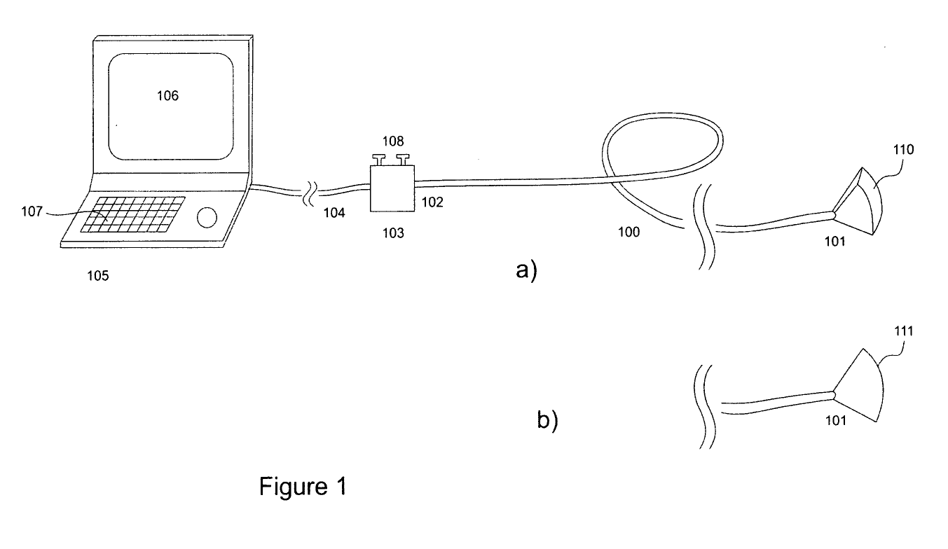

The invention relates to an ultrasound real time imaging system, which in a typical embodiment is composed of the components shown in FIG. 1, where 100 shows an elongated imaging probe with a distal imaging tip 101 and a proximal end 102 that is connected to an utility console interface 103. The imaging ultrasound beam is transmitted from the distal tip of the probe and is for 3D imaging enabled to be scanned within a volume region 110 to be imaged, and for 2D imaging can be enabled to be scanned within a sector surface 111. The utility interface further connects via the cable 104 the probe signals to an ultrasound imaging instrument 105. The imaging instrument has an image display screen 106 for visualization of the images and also other information, and a key board interface 107 for user control of the instrument.

In this particular embodiment, the imaging probe 100 is a particularly flexible catheter probe for example allowing double curving of the probe, which has advantages for imaging inside tortuous vessels and the heart cavities. In other applications, the probe can be much less flexible, close to stiff, for example in minimally invasive surgery where the probes would be inserted through a trocar. For the flexible probe, one often would stretch wires along the periphery of the probe, where the wires can systematically be manipulated by control organs 108 at the utility interface 103 for flexing the tip of the probe in one or two directions.

The invention specially relates to methods of scanning the ultrasound beam within the volume 110 of 3D space and surface 111 for 2D imaging, from the distal tip of such an elongated probe.

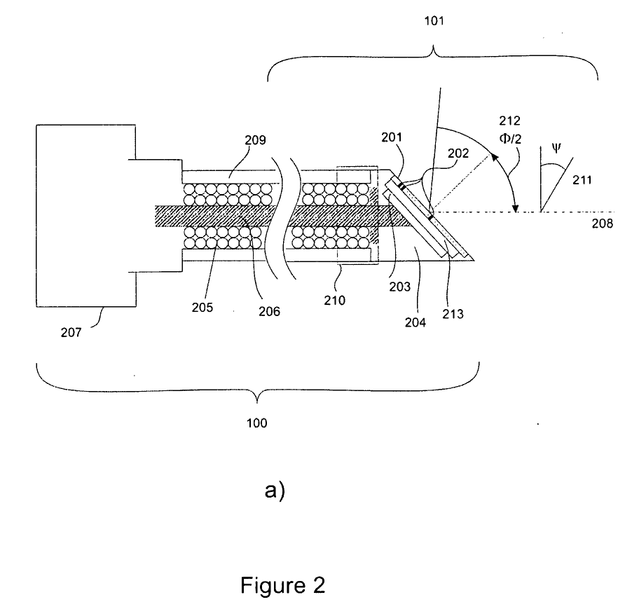

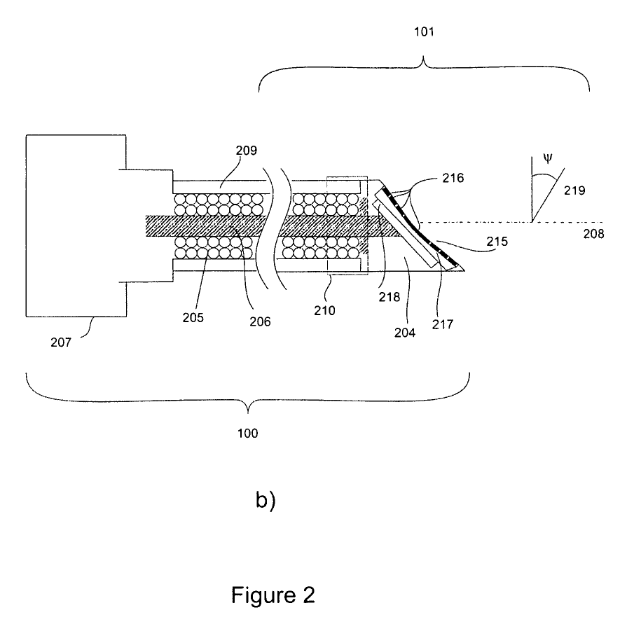

FIG. 2 shows a first example embodiment according to the invention of the distal tip 101 of such an elongated probe 100. FIG. 2a shows a solution for 3D imaging where 201 shows an array transducer that allows electronic direction steering of the ultrasound beam, while FIG. 2b shows simplifications for 2D imaging along a conical surface where the array is modified to a single element or annular array transducer 216. The mechanical scan mechanism and catheter design in other respects are the same for the 2D and 3D imaging.

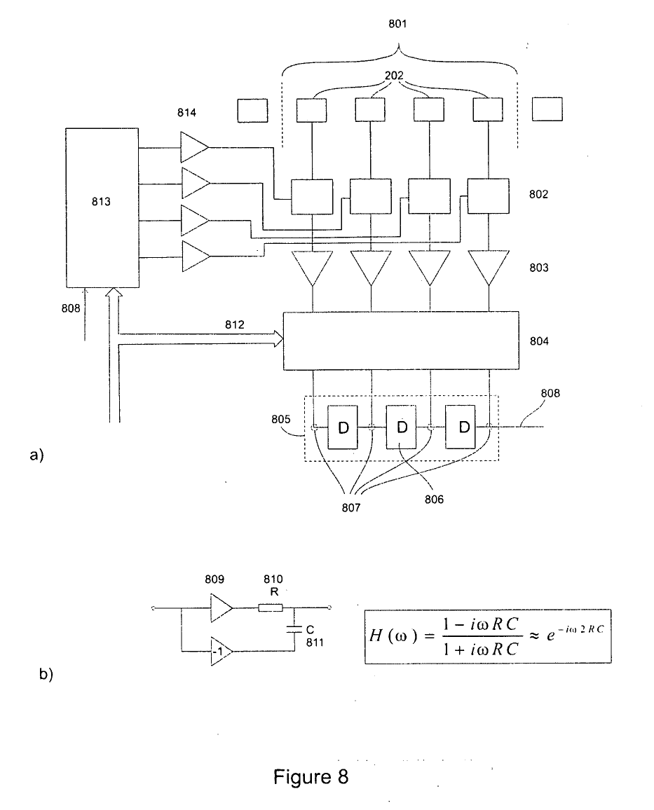

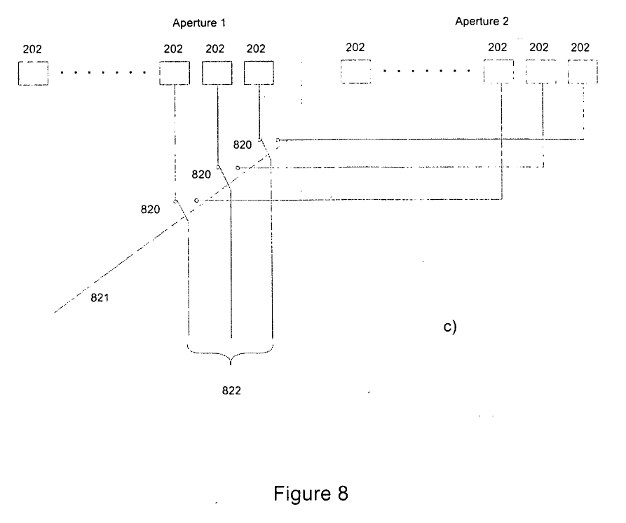

For 3D imaging, the array 201 typically can be a linear phased array transducer with a set of array elements 202, or the array can also for example be a switched array or a curved switched array. The array elements 202 are electrically connected to an electronic circuit 203, with an acoustically isolating material (backing material) 213 between the array and the circuit, to avoid ringing acoustic pulses from the back side of the array. The circuit 203 typically contains receiver amplifiers with switching circuits between transmit and receive of the ultrasound pulses. In some embodiments it can also contain steerable or selectable delay circuits of the receive element signals to combine the signals from neighboring elements in sub-aperture groups into a reduced number of sub-aperture signals, so that the connection of the signals between the array and the utility console can be obtained by less number of wires than the total number of elements in the array aperture, as described in relation to FIG. 8a. When the imaging object has limited movement velocity, the number of wires between the imaging tip and the external imaging instrument can also be reduced with synthetic aperture techniques, as further described below in relation to FIG. 8c. This is important to reduce the diameter of the catheter.

The array 201 and the circuit 203 are mounted in an array holder unit 204 that is connected to a flexible rotation cable 205 typically made of double helix spun wires, like a speedometer wire used in cars. The rotation cable 205 has a core of electric cable wires 206 that connects the array and circuit to the external utility console 103, as shown in FIG. 1. The wire is on the distal end connected to a motor 207 in the utility console, and transmits the motor rotation to rotation of the transducer array 201 around the cable axis 208. The rotating cable would typically be covered with a plastic sheath 209, but this sheath could in some embodiments be left out. One should note that in some embodiments, the electronic circuit 203 can also be left out, and the cable wires 206 would then connect directly to the array elements 202.

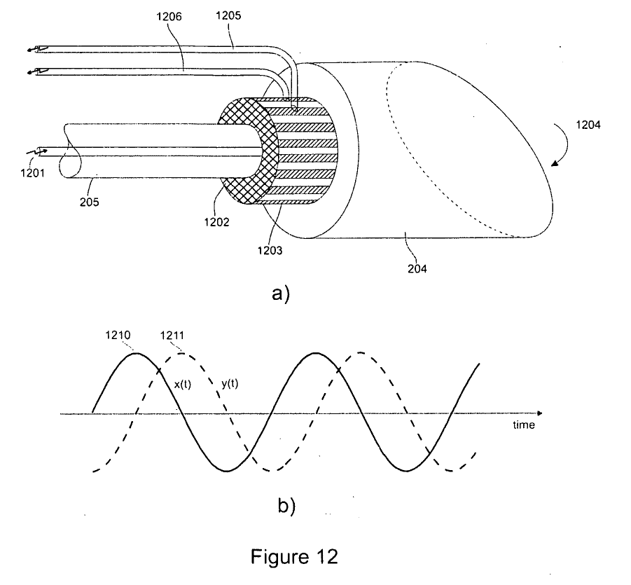

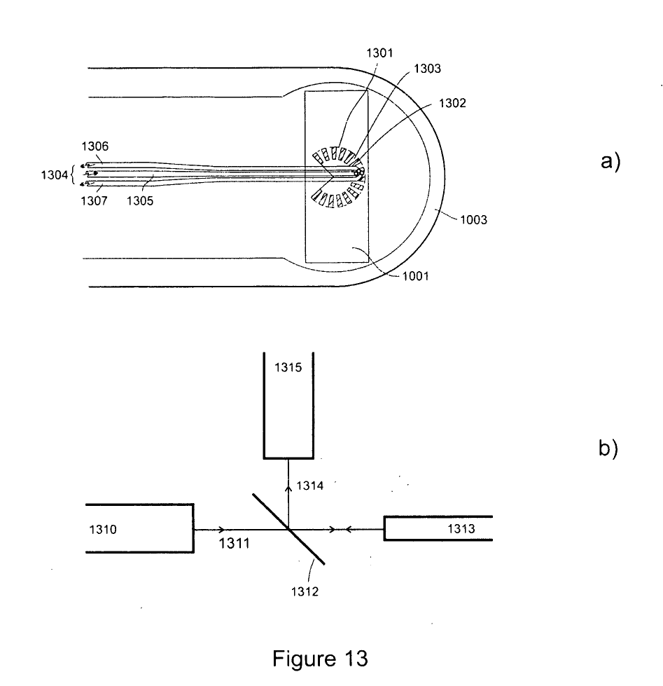

For accurate sensing of the angular direction of the array, a position sensor 210 would typically be mounted at the probe tip to measure the rotation ψ, indicated as 211, of the array holder 204 and array 201 in relation to the catheter sheath 209. This position sensor could typically be of optical types like described in FIGS. 12 and 13, but other methods like electromagnetic angular position sensors could also be used. Accurate monitoring of the angular direction of the array is used in the image reconstruction to minimize the effect of variable angular rotation differences along the cable 205 from the proximal to the distal end, as these differences might vary with rotation angle and bending of the probe.

Other position sensors that relate the rotation to a more global reference, like the patient surface, for example using electromagnetic induction, can also be used. Such sensors are well known to any-one skilled in the art, and would typically be used to merge ultrasound images from different locations of the probe tip, or relate the ultrasound images to images obtained with other modalities like XRay or MR images, or priorly taken ultrasound images, or a treatment tool like a surgical tool, or an ablation tool, etc. The invention also covers embodiments where the rotation of the array holder 204 is mounted directly to a rotating motor at the tip of the array, for example an electric motor or a hydraulic motor.

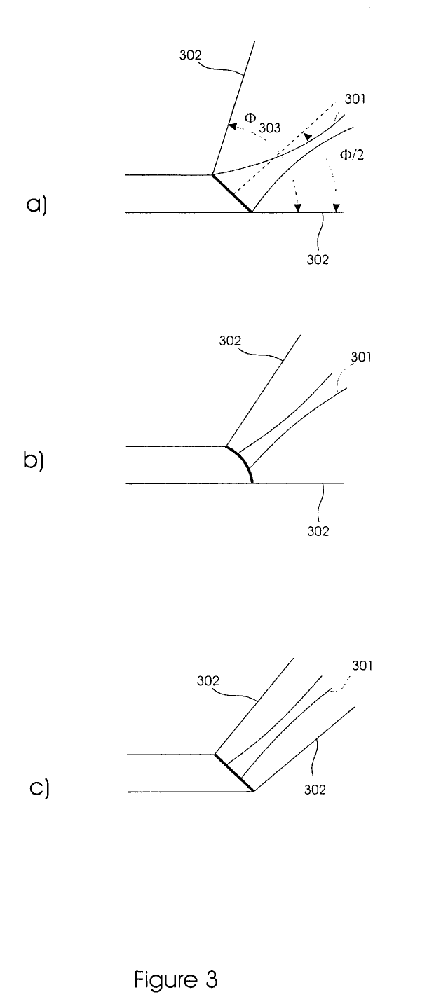

With a linear array embodiment of the array 201, one can obtain electronic direction steering of the ultrasound beam in a sector format with phased array operation as illustrated in FIG. 3a. The full opening angle of the sector scan is ( ), indicated by 303. Beam steering in a weakly opened sector with a switched linear curved array as illustrated in FIG. 3b can also be used. With wide band transducers, for example as shown in U.S. Pat. No. 6,645,150, or U.S. Pat. No. 6,761,692, one could use an array as a phased, sector steered array in a low frequency band where the element pitch is ˜λ/2, where λ is the ultrasound wave length in the tissue, and as a switched array in a high frequency band where the element pitch is ˜λ. Beam steering in a rectangular format with a switched linear array as illustrated in FIG. 3c is then interesting for high frequencies.

In all of these Figures 301 indicates the ultrasound beam for a particular beam direction, and 302 indicates the boundaries of a typical 2D image format, which is obtained with electronic scanning of the beam.

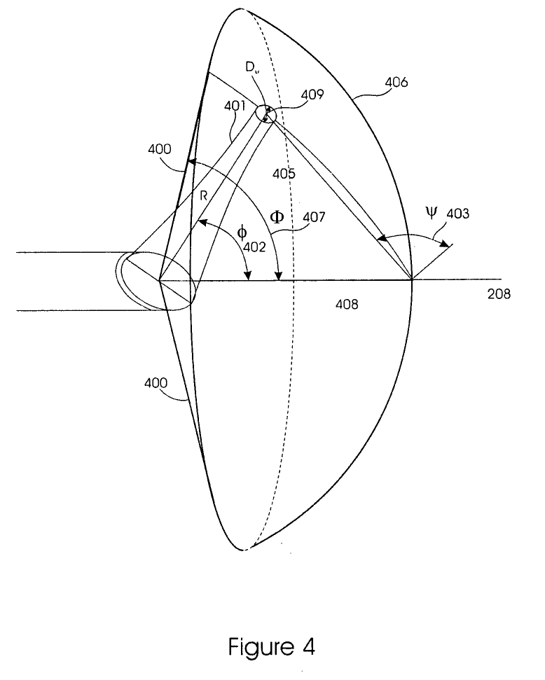

For 3D imaging, the array is rotated around the axis 208, as illustrated in FIG. 4. This Figure illustrates a typical beam 401 obtained with simultaneous electronic steering of the beam with a phased array in the 2D sector format in FIG. 3a, and mechanical rotation of the array. The phased array beam steering provides a beam angle φ (402) relative to the rotation axis of the array. The mechanical rotation of the array gives a beam angle ψ (403) relative to a reference.

The combined electronic and mechanical direction steering of the beam, allows collection of ultrasound backscatter data from a sub-spherical volume region with boundaries 400 determined by the 2D sector 405 and the spherical range of the 2D image 406. With an opening angle Φ (407) of the 2D sector, one could ideally mount the array with a skewed angle to the probe axis 208, so that the normal of the array forms an angle Φ/2 to the probe axis, shown as 212 in FIG. 2. The 3D beam along the probe axis is then obtained with steering of the beam to the outer right side 408 of the 2D sector, and this beam direction can be obtained with all mechanical angular directions ψ of the array, due to the degeneration of the spherical to rectangular coordinate transform for this polar direction. As the ultrasound beam has a certain width determined by the beam focusing and the diffraction, one then can shoot less 2D beams in the directions for small values of φ, than for large values of φ.

Hence, all 2D sectors contain ultrasound beams with large angles φ, while smaller values of φ are only found in selected 2D sectors, so that the beam density is kept approximately constant in the real 3D space. By example, with a lateral sampling width D_{ψ}, of the beam in the ψ direction at the maximal range R, indicated as 409 in FIG. 4, the number of beams required as a function of φ for adequate 3D sampling of the image at this angle is

N(φ)=2πR sin φ/D_{ψ} (1)

Hence, the fraction of the rotating 2D sectors that has beams at an angle φ is

η(φ)=N(φ)/N(Φ)=sin φ/sin Φ (2)

With a sampling coverage area A_{b} of the beam at the image range R, given by sampling criteria on the 3D image, one would cover the image area with N beams where

N_{3D}(Φ)=2π(1−cos Φ)R²/A_{b} (3)

Typical values are A_{b}=3 mm², R=50 mm, and Φ=π/2, which gives N_{3D}(π/2)˜5200. With 70 μsec per beam, it takes ˜370 msec to collect a full 3D frame, i.e. 2.7 3D frames per second. Transmitting a wide beam, and processing 2, 3, or 4 receive beams with small angular differences in parallel for each transmit beam in a known manner, the 3D frame rate can be increased to ˜5, 8, or 10 3D frames per second. The frame rate can be further increased by reducing the 3D opening angle Φ, where a reduction to Φ=π/3 reduces the number of beams to N_{3D}(π/3)=2600 with an increase in the 3D frame rate to 5, 10, 16, and 20, and Φ=π/4 reduces the number of beams to N_{3D}(π/4)=1500 with an increase in the 3D frame rate to 9, 17, 28, and 38 3D frames per sec for 1, 2, 3, and 4 parallel receive beams respectively. Due to complexities with the mechanical rotation, one would prefer a 3D frame rate 10-20 per sec, which can be achieved with 2 parallel receive beams up to an opening angle of π/3, which is a highly adequate value.

An adequate diameter of such a catheter probe for intra-cardiac echo applications (ICE), is 3 mm. With Φ=π/3, one can use a skewed mounting of the array where the array normal forms an angle π/6 relative to the forward rotation axis 208. This gives a maximal aperture diameter of the array in the 2D azimuth direction of 3/cos(π/6)=3.4 mm. With 64 elements in the phased array, the element pitch becomes ˜50 μm, which allows ultrasound wave lengths down to 100 μm corresponding to frequencies up to 15 MHz.

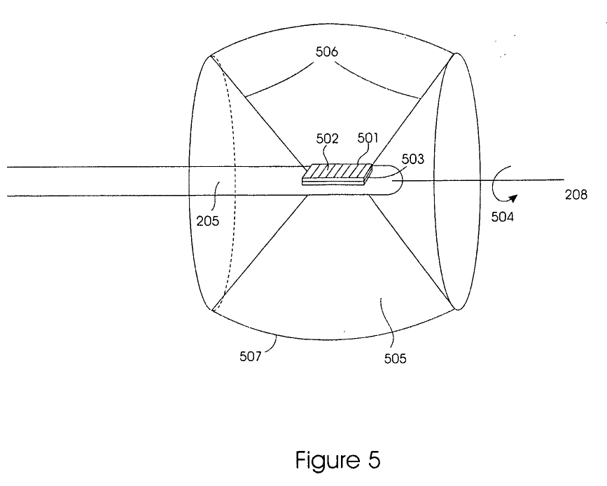

A variability of applications using real time 3D imaging, can benefit from different forms of the 3D scanning region, where for example FIG. 5 shows a modification compared to FIG. 2 of the rotating array, where in FIG. 5 the array 501 with elements 502 and an optional electronic circuit 503, is mounted on the side of the flexible, rotating wire shaft 205. Rotating the shaft around its axis 208 for example in the clockwise direction 504, produces a “donut” like 3D volume scanning region of the ultrasound beam 505 limited by the sector opening lines 506 and the depth range 507. Other arrays like switched linear or curvilinear arrays can also be used. It is also clear that for various purposes, the array can according to the invention be mounted with a variety of angles relative to the rotation axis 208, to provide a variety of 3D scanning regions for a variety of applications.

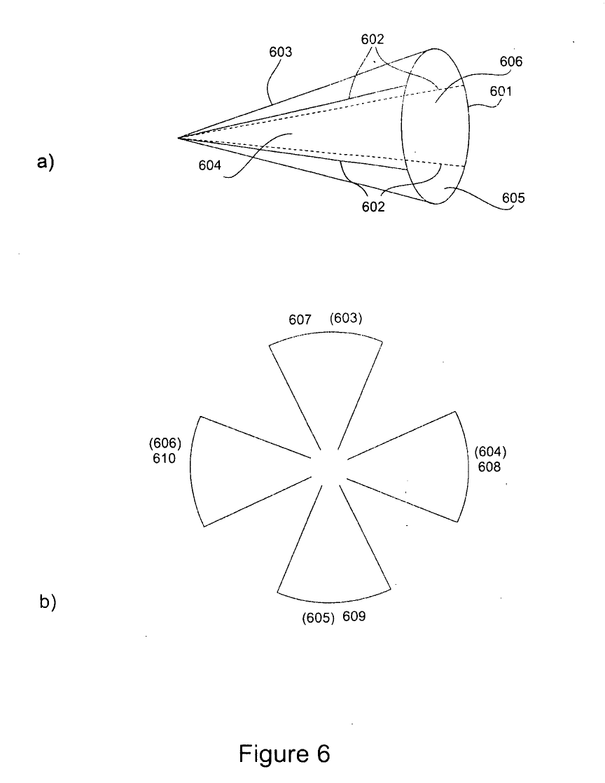

For 2D imaging, the electronic direction steering array 201 could typically be exchanged with a single element, fixed focus transducer, or an annular array transducer as 215 in FIG. 2b with annular elements 216. The 2D transducer elements is mounted on a backing material 217 and annular array elements can also be connected to an integrated circuit 218 with receiver amplifiers and also beam forming circuits for electronic focus steering, where for example methods and implementations according to U.S. Pat. No. 6,540,677 could be used. The mechanical scanning system is otherwise the same as for the 3D imaging probe, which gives a lateral scanning of the ultrasound beam across a conical surface illustrated as 601 in FIG. 6a, where the opening angle of the conical surface is given by the direction y of the transducer surface indicated as 219 in FIG. 2b.

An example of visualization of the 2D conic image data on a flat screen, is shown in FIG. 6b. The physical, conical surface 601 across which the beam is scanned, is divided for example along 4 radial lines 602 to be separated into 4 surface regions 603, 604, 605, and 606. These surface regions are then projected onto the plane sectors 607, 608, 609, and 610 displayed on the image screen in the same sequence. The images are typically shown as grey scale images for the amplitude of the reflections that gives a tissue image, or in a color scale for movement velocities of the object, according to well-known principles.

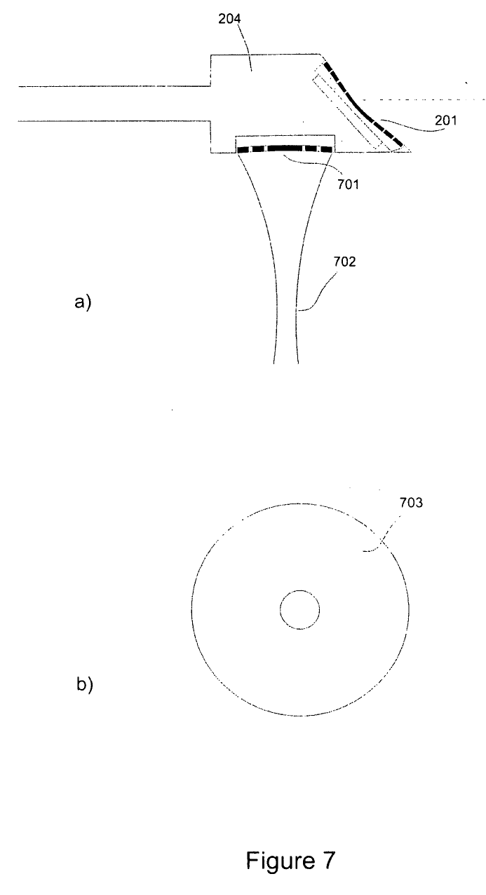

For various applications, for example for measurement of a vessel cross section or observations of the cardiac valves, it is advantageous in addition to the forward cone to show a cross sectional image around the probe tip. This can be achieved as shown in FIG. 7, which shows a similar probe tip as in FIG. 2, but with an added transducer array 701 with a beam 702 at the circumference of the rotating array holder unit 204. This transducer can again be a single element transducer or an annular transducer array, similar to the forward looking array 201. The 2D image would then be displayed as 703 on the screen, typically together with the forward looking image cone as in FIG. 6. Due to the angular difference between the forward and transverse looking beams from the arrays 215 and 701, one could transmit the image pulses for these arrays at the same time, and record the back scattered signals in parallel. However, this will generate some acoustic cross talk noise between the two beams, and also requires parallel electronics to operate the arrays. Allowing for some reduction in image frame rate, one would rather operate the two arrays with interleaved time multiplexing, transmitting each second pulse on array 215 and the other pulses on array 701.

As mentioned above, it is advantageous to use electronic circuits close to the array, for example with amplifiers to maintain signal to noise ratio in reception, but especially with some local sub-aperture beam forming that allows reduction in the number of independent signals that have to be transmitted along the probe. Example embodiments of such sub-aperture beam forming is shown in FIG. 8a, where a group 801 of the array elements 202 forms one of the sub-apertures. In this particular example, the number of elements in the sub-aperture group is 4, but other numbers of 2, 3, etc. can be used.

The signals from each element are fed to transmit/receive switches 802 that in receive mode feed the signal to receiver amplifiers 803. The outputs of the receiver amplifiers are in this example embodiment of sub-aperture processing fed to a cross-point switch 804 that connects the element signals to a summing delay line system 805 with a set of delay elements 806, so that any of the signals can be connected to any delay-point 807. The output 808 of the delay line system is then fed via the cable 206 to the external imaging instrument where the sub-aperture signals are combined to a complete beam, or several parallel beams, according to known methods.

An example embodiment of a delay cell is shown in FIG. 8b, where 809 indicates a differential amplifier, and the signal delay is given by the resistor 810 and capacitor 811. The transfer function of this delay cell is

$$H ( ω ) = \frac{1 - iω RC}{1 + iω RC} = {e}^{- i 2 {tan}^{- 1} ω RC} \approx {e}^{- iω 2 RC} \qquad ( 4 )$$

which when □<1/RC gives a signal delay □=2RC for each cell. The actual delay for each element signal is then determined by which summing point 807 the signal is fed into the delay line structure 805, determined by the set-up of the cross point switch 804 that is done via the sub-aperture control bus 812.

The transmit beam of the sub-aperture can in its most general form be generated by a sub-aperture transmit beam former 813 that feeds a set of element driver amplifiers 814 with transmit pulses that have intermediate delays given by the sub-aperture transmit beam former 813 and set up by the sub-aperture control bus 812. The transmit trigger pulse for each sub-aperture transmit beam-former is conveniently transferred on the receive line 808 for each sub-aperture, to limit the number of wires connecting the catheter tip and the imaging instrument.

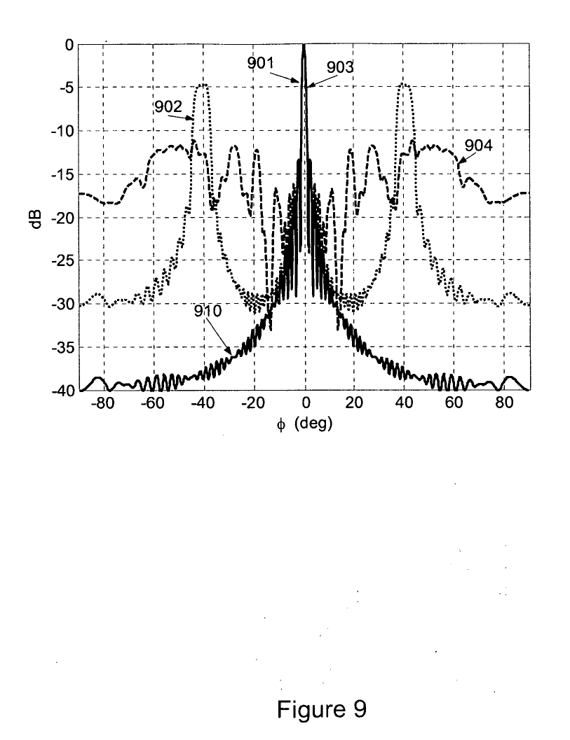

The sub-aperture transmit beam-former can in some applications be left out, where only a sub-set of the elements in the sub-aperture, for example 1 or 2 elements, are used to transmit at each sub-aperture. In these cases, the transmit beam would get grating lobes as illustrated in FIG. 9 for a wide band transmit pulse. This Figure shows the amplitude of the far-field beam profile of the transmitted beam as a function of azimuth angular direction from the beam center axis. In the examples of FIG. 9, each sub-aperture has 3 elements, and a single element is used to transmit from each sub-aperture. 901 shows the main lobe and 902 shows the grating lobes of the beam where the transmit elements have the same position within each sub-aperture (periodic position of the transmit elements). 903 shows the main lobe of the beam where the transmit elements have a close to random variation of their position within each sub-aperture, and 904 shows the side lobes for this random location. We notice that with the random variation of the transmit elements, the sharp peak grating lobe disappears, and we get a more even leveled side lobes which are higher than the side lobes for the transmit elements with a fixed position in each sub-aperture. The sidelobes close to the main lobe of the sub-set transmit apertures are close to the sidelobes for the fully sampled aperture, while the sidelobe/grating lobe level 904 at large distance from the main lobe is ˜20 dB higher than for the fully sampled aperture 910.

The transmit beam grating lobes are also reduced by focusing of the beam. Their effect on the image is further reduced since the receive beam which uses the full sub-apertures is missing these grating lobes, hence attenuating the back scattered signal from these lobes. However, the transmitted grating lobes would generate some noise in the image, and lowest noise images would be obtained with a full sub-aperture transmit beam former, illustrated as 813 in FIG. 8a.

When the imaging object has limited movement velocity, one can also reduce the number of wires connecting the imaging tip and the external instrument with a synthetic aperture method, as illustrated in FIG. 8c. The total array aperture is in this Figure divided in the middle into two, equal groups, Aperture 1 and Aperture 2. The array elements 202 are pair wise from each group connected to 2 to 1 multiplexers 820, which through the control signal 821 connect either the elements from Aperture 1 (as shown in the Figure) or Aperture 2 to the wires 822 that connects through the probe to the external imaging instrument. Switching the multiplexers 820, image beams are in an alternating sequence collected from Aperture 1 and Aperture 2, and then the RF signal for the two beams from Aperture 1 and Aperture 2 are combined with the Synthetic aperture technique known to anyone skilled in the art, into a single beam focused at all depths with focus width determined by the full aperture.

Dynamic focusing of the beam in the elevation direction can be achieved by dividing the array elements in the elevation direction, and feeding the element signals to a sub-aperture beam former like exemplified in FIG. 8a for each azimuth position of the elements. The outputs of the elevation focusing beam formers from neighboring azimuth elements, could then in turn be fed to azimuth sub-aperture beam formers as described. For the elevation focusing, one would only need small delays, as there is no large electronic direction steering of the beam in the elevation direction. Small electronic direction steering is highly interesting to use parallel receive beams in the elevation direction also, to increase the frame rate, in a manner known to anyone skilled in the art.

For the elevation focusing of the transmit beam, one would in most situations avoid the transmit beam former 813 of FIG. 8a and transmit with a central group of elevation elements with a fixed pre-focusing. This gives an adequately elevation focused transmit beam, as the receive focusing provides the dynamic elevation focusing. With parallel elevation beams, the elevation width of the transmit beam must also be sufficiently high.

When the object has limited movement velocity, one can also obtain depth adjusted focusing of the image beam by a synthetic aperture linear combination of the received RF signal of the beams with same azimuth direction from a group of neighboring elevation scans.

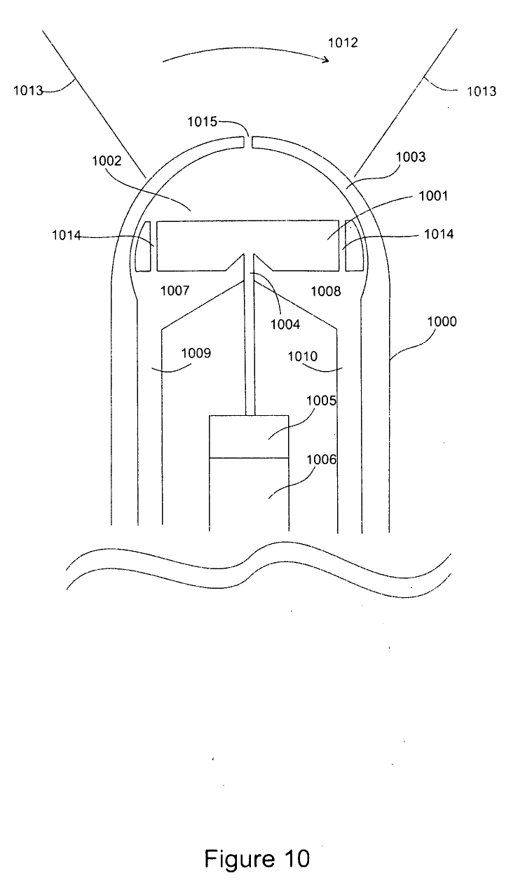

Yet another embodiment for the mechanical beam scanning both for 2D and 3D imaging according to the invention, is shown in FIG. 10, where 1001 shows the combined array and integrated circuit assembly that is enclosed in a sub-spherical dome 1003. For 3D imaging the array would provide electronic direction steering of the ultrasound beam in one direction, as for example shown in FIG. 3, while for 2D imaging the array could typically be a single element transducer with fixed focus or an annular array as described in relation to FIG. 2b. The assembly 1001 is connected to a flexible member 1004 that locates the assembly in the middle of the dome and also feeds electric signal wires from the array and electronic circuit to the imaging instrument. The member 1004 can for example be made as a printed flex circuit or similar structure. The signal wires can connect to a more convenient type of cable 1006 at the interface 1005 to be fed throughout the probe to connect to the utility console 103 of FIG. 1.

The probe in this example embodiment contains two hydraulic channels 1009 and 1010 that can inject or remove fluid from the chambers 1007 and 1008, that are separated by the flexing member 1004. In normal scanning operation, the interior compartments 1002, 1007, and 1008 are filled with a fluid, preferable water with physiological composition. Injecting fluid through the tube 1009 into compartment 1007 while removing fluid through tube 1010 from compartment 1008 causes the array/circuit assembly 1001 to rotate in the clockwise direction indicated by the arrow 1012. The opposite rotation is obtained by injecting fluid through tube 1010 into chamber 1008 while removing fluid through tube 1009 from chamber 1007. Using valves at the distal end of the channel, one can also construct designs, where one fluid channel feeds continuously fluid out to the array, and the fluid is either dumped out of the probe at the tip, or returned to the proximal end of the probe through a separate channel.

For simplified filling of the chambers 1002, 1007, and 1008 with fluid, without introducing air bubbles, a continuous forward filling with fluid is obtained by the channels 1014 that feeds fluid from the compartments 1007 and 1008 into the compartment 1002, while the channel 1015 feeds fluid from the compartment 1002 to the outside front of the probe dome. This continuous flow of fluid to the front of the dome, improves acoustic contact between the dome and the object contact surface, or can spill into the blood when the probe is inserted into a blood-filled region. In other embodiments, the draining of the fluid from compartment 1002 can be done through the probe to its proximal, outside end, by a specific channel through the probe from the distal to the proximal end.

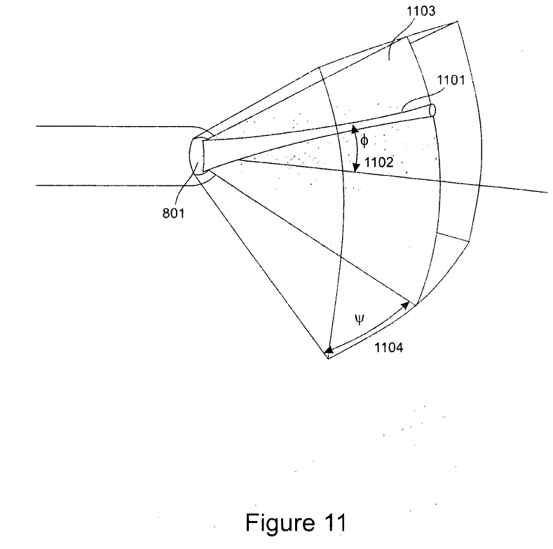

The probe is on its proximal end connected electrically and hydraulically to the utility console 103 of FIG. 1, which for this embodiment also contains a hydraulic pumping and control system that injects or removes fluid through the channels 1009 and 1010 and provides the wobbling motion of the array assembly 1001. The array can typically be a linear phased array with a 2D azimuth scan normal to the wobbling direction which is in the elevation direction of the 2D scan. This provides a three-dimensional scanning of the ultrasound beam as illustrated in FIG. 11, where 1101 indicates an ultrasound transmit/receive beam for the array/circuit assembly 1001 with azimuth angle φ illustrated as 1102 within a 2D sector 1103. The wobbling of the array/circuit assembly 1001 provides a steerable elevation angle ψ of the 2D sector and the beam indicated as 1104. It should be clear that this hydraulic wobbling mechanism also allows for mounting of the array so that the wobbling axis forms an angle other than 90 deg to the probe tip axis, to adjust the detailed location of the 3D imaging region in relation to the probe tip axis to the particular application at hand.

With a sampling coverage area A_{b} of the beam at image range R, azimuth scan opening angle of Φ and elevation scan opening angle of Ψ, one would with the scanning method described in FIGS. 10 and 11 require N_{3D} beams where

N_{3D}=ΦΨR²/A_{b} (5)

With Φ=Ψ=π/2, R=50 mm and A_{b}=3 mm², a total number of beams to cover the region is N_{3D}=2056. With 70 μsec per beam, it takes 144 msec to capture the image, giving ˜6 3D frames per second. Using 2, 3, and 4 parallel beams increases the 3D frame rate to 12, 18, and 24 3D frames per second with this opening angle. Reducing the opening angles to Φ=Ψ=π/3, decreases the required number of beams to N₃D=914, increasing the 3D frame rate to ˜15, 30, 45, and 60 with 1, 2, 3, and 4 parallel receive beams. This scan method could hence conveniently be used with down to one single parallel receive beam, where the high 3D frame rates are obtained by decreasing the 3D scan opening angle below (π/2)².

For 2D imaging, the mechanical wobbling of the array assembly 1001 would provide a lateral sector scanning of the ultrasound beam that would provide a 2D sector image of tissue structures and scatterer velocities and displacements that is displayed according to known methods. It should also be clear from FIG. 10 that the middle position of the array assembly 1001 could also be rotated to provide sector scans with an angle to the probe tip. The assembly could also be rotated to also provide sector scans that have a cross sectional position to the probe axis.

To avoid geometric distortions of the image in the direction of the mechanical scan, one can conveniently use an angular position sensor of the moving array/circuit assembly at the tip of the probe. Such position sensor can be based on optical or electromagnetic principles according to known methods, and for sake of example FIG. 12 illustrate an optical position sensor for the rotating scan system of FIGS. 2, 5 and 7, and FIG. 13 illustrate an optical position sensor for the wobbling scan system in FIG. 10.

FIG. 12a shows the rotating array holder 204 with the rotating drive cable 205, that rotates the array in the direction indicated by 1204. The rotating drive cable contains in this example embodiment also an optical fiber 1201 that feeds light into a transparent sub-part 1202 of the array holder. The surface of the sub-part 1202 is partly covered with a light inhibiting film at the end face and also at grating lines 1203 in a periodic pattern along the circumference of 1202 that inhibits light to shine out through the circumference, while between the grating lines the light is allowed to shine through. The distance between the grating lines is equal to the width of the grating lines within the accuracy of the manufacturing.

Two optical fibers 1205 and 1206 picks up light that shines through the circumference of 1202 and feeds the light back to the instrument where it is converted to electrical analog signals by for example photo transistors and subsequently converted to digital form for processing to accurately detect the rotational angle of the array holder 204. Example signals after the phototransistors for the two fibers are shown in FIG. 12b where 1210 shows a typical signal x(t) from fiber 1205 and 1211 shows a typical signal y(t) from fiber 1206. Due to spread of the light, the signals are close to sinusoidal in shape. The two fibers 1205 and 1206 have a distance between each other close to ¼ of the period of the grating lines, which gives close to 90 deg phase lag of y(t) in relation to x(t). An accurate resolving of the rotational angle ψ can then for example be found by the following relation

ψ(t)=F{x(t),y(t)} (6)

where for many applications F can be approximated by the inverse tangent as

ψ(t)=F{x(t),y(t)}=tan⁻¹{y(t)/x(t)} (7)

A similar optical position sensor for the wobbling system in FIG. 10, is shown in FIG. 13a, where 1001 shows the array holder within the dome 1003. In this example embodiment, a variable reflectance grating 1301 composed of stripes 1302 with high reflectance periodically arranged with stripes 1103 of low reflectance. A triple optical fiber system 1304 containing one fiber 1305 for shining light onto the reflectance grating, and two fibers 1306 and 1307 for transmitting the light reflected from the grating to the instrument. The reflected light is detected and digitized in the instrument as for the position sensor in FIG. 12a. The distance between the pickup areas of fiber 1306 and 1307 is ¼ of the grating period, so that the signals in the two fibers 1306 and 1307 produces signals x(t) and y(t) as in FIG. 12b, which is further processed to resolve the angular position of the array holder similar to Eqs. (6,7).

In FIG. 12a is shown a position sensor with a transmitting grating, while it is clear to any one skilled in the art that a reflecting grating could equally well be used similar to the sensor in FIG. 13a, for which sensor one could also use a transmitting grating.

With two fibers that collects light that is 90 deg out of phase with each other (quadrature phase) one is able to resolve the direction of rotation. If one knows the rotation direction, it would be sufficient to have a single fiber for the reflected light, however, the conversion from light intensity to angle would be simplified by the use of two light signals with quadrature phase relationship.

The same fiber can also be used for transmitted and reflected light using for example a transmitting mirror as shown in FIG. 13b. The light source 1310 shines a light beam 1311 through a transmitting mirror 1312 so that the light enters the fiber 1313. The light reflected at the distal end of the fiber will then come out of the tip and be reflected at the mirror 1312 so that the reflected light is separated into the beam 1314 that hits the detector 1315 and is converted to an electrical signal and digitized.

Other methods of angular position sensing can be based on electromagnetic methods of measuring the array angle in relation to the tip, and also in relation to the external world.

The frequency and bandwidth of the arrays can be selected freely within the scope of the invention. Using wide band or multi-band transducers as described in U.S. Pat. No. 6,645,150 or based on ceramic films as described in U.S. Pat. No. 6,791,692, one can operate the ultrasound transducer both in a low frequency band for an overview image with large penetration, and in a high frequency band for a short range image with improved resolution. The overview image could for example be used to guide one's way in the cardiac chambers to move the probe tip close to an electrophysiology ablation scar, and then evaluate the scar with the high resolution short range image. Similarly could the long range image be used to get an overview of the movement of native heart valves to evaluate best procedure for valve repair or valve replacement, while the short range image can be used to evaluate details in valve morphology. The probes can also be used with the new dual frequency pulse technique described in PCT Application PCT/NO20005/00278. Example array designs for such applications are given in U.S. Provisional Patent Application of July 26 by B. A. J. Angelsen et. al. With such dual frequency techniques the probes could be used for improved imaging of ultrasound contrast agent micro-bubbles, micro-bubbles produced by extracorporal blood oxygenators or in decompression situations, micro-calcifications found in tumors and atherosclerotic plaque, and assessment of tissue properties.

It is also expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment as a general matter of design choice. It is the intention, therefore, to be limited only as indicated by the scope of the claims appended hereto.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed:

1. An ultrasound imaging probe with a distal imaging end to be inserted into a body and a proximal end, opposite along the probe to said distal end, to be connected to an external ultrasound imaging instrument outside said body, where
said distal imaging end contains an ultrasound transducer array capable of electronic steering an ultrasound beam in a 2D azimuth plane, and in addition
said transducer array being mechanically moveable at said distal imaging end in the elevation direction at close to right angle to said 2D azimuth plane, so that through combined electronic steering of the beam in said 2D azimuth plane and mechanical steering of said array in said elevation direction, the ultrasound beam can be steered in selected directions in a 3D image region around the distal probe end.

2. An ultrasound imaging probe according to claim 1, where said mechanical elevation scanning of the ultrasound beam is obtained by rotating said array around the long axis of said probe.

3. An ultrasound imaging probe according to claim 2, where said array is mounted to a rotation cable that is rotated by a motor connected to said proximal end of the probe, so that rotation of said array by said motor via said cable provides said elevation steering of the array.

4. An ultrasound imaging probe according to claim 3, where said rotation cable is a dual helix wire spun around an electrical cable that connects the signals from said array to said external imaging instrument.

5. An ultrasound imaging probe according to claim 2, where
the beam position in each azimuth scan is selected as a function of the azimuth angle between the beam and the rotation axis of the array, and the beam positions in neighboring azimuth scans,
so that
the beam density is approximately constant in 3D space over the whole 3D image region.

6. An ultrasound imaging method utilizing a probe according to claim 2, where the azimuth direction of the beam is kept constant for at least a partial rotation of said array so that the beam for said at least partial rotation of the array is swept across a conical surface in the forwards direction of the probe to provide ultrasound images along said conical surface, and said ultrasound images are broken into one or more portions where each of said portions is displayed as a 2D image on an image screen.

7. An ultrasound imaging method according to claim 6, where the array of said probe is substituted with a fixed focus single element transducer or an annular array transducer with a beam with the given angular direction in relation to the probe axis.

8. An ultrasound imaging method according to claim 7, where in addition to said transducer or annular array a 2^{nd} transducer or annular array is mounted to the rotating assembly where said 2^{nd} transducer or annular array provides ultrasound beams close to normal angle to the probe axis to provide cross sectional 2D images to said probe axis in addition to said 2D images across said conical surface.

9. An ultrasound imaging probe according to claim 1, where said mechanical elevation steering of the ultrasound beam is obtained by wobbling of the array in a back and forth manner.

10. An ultrasound imaging probe according to claim 9, where said wobbling of the ultrasound array is obtained by hydraulic pumping of fluid through at least one channel in the probe, the pumping mechanism being connected to the proximal end of the probe.

11. An ultrasound imaging probe according to claim 10, where the probe hydraulic fluid fills the space around the array in the distal probe end to function as an acoustic transmission fluid, and the distal end contains one or more draining channels of the hydraulic fluid so that a continuous flow of fluid around the array is obtained to remove possible gas bubbles in the fluid around the array.

12. An ultrasound imaging probe according to claim 11, where at least one draining channel leads said hydraulic fluid to the exterior of said distal probe end.

13. An elongated ultrasound imaging probe according to claim 1, where said array is one of a linear phased array, and a curved linear switched array, and a linear switched array.

14. An ultrasound imaging probe according to claim 13, where said array is used in a phased array mode in a lower frequency range, and in a switched array mode in a higher frequency range.

15. An ultrasound imaging probe according to claim 13, where the elements of said arrays are divided in the elevation direction for one or both of
transmission of a wide beam with multiple parallel receive beams in the elevation direction within the transmit beam, to increase the frame rate with 3D imaging, and
electronic steering of the ultrasound beam focus also in the elevation direction.

16. An ultrasound imaging probe according to claim 13, where depth adjusted focusing of the imaging beams in the elevation direction for each azimuth position of the beams is obtained by linear combination in the elevation scan direction of the RF signal in a group of neighboring elevation scans.

17. An ultrasound imaging probe according to claim 13 or 14, where multiple receive beams within the transmit beam in the elevation direction for each azimuth position of the beams is obtained by linear combination in the elevation scan direction of the RF signal in a group of neighboring elevation scans.

18. An ultrasound imaging probe according to claim 1, where said distal end of the probe contains integrated circuits with receiver amplifiers for high sensitivity imaging.

19. An ultrasound imaging probe according to claim 1, where said distal end of the probe contains integrated circuits with receiver amplifiers and delay circuits to combine received signals from neighboring elements into sub-aperture signals, so that the number of wires connecting said integrated circuit and the external imaging instrument is reduced compared to the number of elements in said array.

20. An ultrasound imaging probe according to claim 19, where a subgroup of the array elements are used to form the transmission beam.
